(19) United States
(12) Patent Application Publication
(10) Pub. No.: US 2017/0231839 A1
(43) Pub. Date: Aug. 17, 2017

(54) Disposable Diaper And Cushion Sheet

(71) Applicant: OJI HOLDINGS CORPORATION, Chuo-ku, Tokyo (JP)

(72) Inventor: Izumi TASHIRO, Chuo-ku, Tokyo (JP)

(21) Appl. No.: 15/504,658

(22) PCT Filed: Aug. 5, 2015
(86) PCT No.: PCT/JP2015/072164

§ 371 Date: Feb. 17, 2017

(30) Foreign Application Priority Data
Aug. 20, 2014 (JP) 2014-167491

(51) Int. Cl. A61F 13/515; A61F 13/49

(52) U.S. Cl. CPC A61F 13/515; A61F 13/49011

(57) ABSTRACT

To maintain, in a wide state, the spaces in a ventilation channel formed when a waist expansion member contracts. This disposable diaper has formed therein, on one or both of a front part (1) and a rear part (2), a cushion part (40). The cushion part (40) includes: an inner sheet (41); an outer sheet (42); one or a plurality of waist expansion members (43); and a cushion forming sheet (44). In a state where the waist expansion member (43) is stretched in the width direction, the member is secured between the inner sheet (41) and the outer sheet (42). The cushion forming sheet (44) overlaps one or both of the inner sheet (41) and the outer sheet (42), and is joined thereto so that in the width direction joining portions (51) and non-joining portions (52) are alternatingly continuous. The joining portions (51) are formed by joining-lines (53) which are straight and extend in the longitudinal direction and at least two columns of which are side by side. The space (second space (D2)) between the joining-line columns in one joining portion is narrower than the space (first space (D1)) between adjacent joining portions.

### TECHNICAL FIELD

The present invention relates to a disposable diaper. More specifically, the invention relates to a disposable diaper in which a waist gather having a high cushioning property may be formed in a waist end part of the diaper.

### BACKGROUND ART

A disposable diaper for an infant and a toddler or for an aged person has been known. Various types of disposable diapers are present. For example, a pants-type disposable diaper and a tape-type disposable diaper are known (for example, Patent Literature 1, Patent Literature 2, and Patent Literature 3).

The pants-type disposable diaper is a disposable diaper in which both side portions corresponding to a front body part and a rear body part are joined to each other in advance. Examples of the known pants-type disposable diaper include an integrated disposable diaper (Patent Literature 1) in which an outer packaging body on which an absorbent main body is placed is one body across a rear body part from a front body part, and a separation-type disposable diaper (Patent Literature 2) in which an outer packaging body is separated into a front body part and a rear body part, and an absorbent main body is connected between a front body part outer packaging body and a rear body part outer packaging body such that the absorbent main body is bridged therebetween. In addition, the tape-type disposable diaper is a disposable diaper in which an adhesive tape is attached to both side portions corresponding to a front body part and a rear body part, and the front body part and the rear body part are attached to each other using the adhesive tape at the time of wearing (Patent Literature 3).

In the pants-type disposable diaper, in general, a plurality of stretchable members is fixed in an extended state to a rim of a waist opening formed by attaching the front body part to the rear body part. As these stretchable members tighten the waist circumference of the wearer, the diaper may be worn without slipping down due to movement of the wearer or a weight of excreta. A function for preventing the diaper from slipping down by providing the stretchable members at the rim of the waist opening is important.

Meanwhile, when the diaper is excessively strongly fastened around the waist, the wearer may feel strongly tightened. In addition, when the diaper is strongly fastened, adhesion increases. In contrast, air permeability is degraded.

Herein, there has been a known technology for forming a waist gather to assign an appropriate tightening force to the circumference of the waist of the disposable diaper. In more detail, by fixing a plurality of stretchable members in an extended state between cover sheets included in the circumference of the waist of the disposable diaper, a wrinkle is generated in a cover sheet adjacent to a waist opening when these stretchable members contract. Due to the wrinkle, a waist gather is formed around the waist opening. This waist gather may improve adhesion of the disposable diaper by coming into contact of a skin of the wearer, and thus a fitting feeling around the waist is improved. In addition, the waist gather functions as a cushion for relieving the fastening being done to the wearer due to the stretchable members.

Further, Patent Literature 4 discloses a technology for providing a disposable diaper that has flexibility and a cushioning property in a whole waist gather. This technology related to the waist gather is also referred to as a “flute structure”. The flute structure includes a pair of sheets for fixing a waist stretchable member by interposing the waist stretchable member therebetween, and a sheet for forming a wrinkle (cushion forming sheet) joined to a top of the sheets by intermittent joining portions. In the flute structure, when the waist stretchable member contracts, the cushion forming sheet rises at a portion (non-joining portion) which is not joined. In this way, when a ventilation passage (space) is formed between the cushion forming sheet and the pair of sheets interposing the waist stretchable member therebetween, hardness of the pair of sheets interposing the waist stretchable member therebetween is unlikely to be directly delivered to the skin. For this reason, air permeability, flexibility, and a cushioning property may be assigned to the whole waist gather by forming the flute structure.

### CITATION LIST

### Patent Literature

### Patent Literature 1: JP 2009-254662 A

### Patent Literature 2: JP 2010-233733 A

### Patent Literature 3: JP 2005-323685 A

### Patent Literature 4: JP 2013-070711 A

### SUMMARY OF INVENTION

### Technical Problem

Incidentally, in the “flute structure” disclosed in Patent Literature 4, basically, a joining portion of sheets is formed by joining lines or joining points arranged in one column. For this reason, when the waist stretchable members contract, a ventilation passage (space) is formed between the joining lines of the one column. However, when sheets are joined by joining lines, etc. arranged in one column, ventilation passages, which are formed when the waist stretchable members contract, excessively approach each other. As a result, there has been a problem that the ventilation passages pressed each other, and narrowed the ventilation passages between the sheets.

In addition, in the “flute structure”, in order to widen an interval between ventilation passages, for example, it is considered that the joining lines are formed to be thick, or a plurality of joining points is densely formed, thereby widening a width of the joining portion of the sheets. However, when the joining lines are thick, or the joining points are dense, there is a problem that air permeability is poor in the joining portion, or a tactile property of the joining portion is poor.

In this regard, an issue to be solved by the invention is to improve air permeability and a tactile property of a joining potion while ensuring a wide interval between ventilation passages formed when waist stretchable members contract in a “flute structure”.

### Solution to Problem

As a result of a keen examination of means for solving a conventional problem, the inventors of the invention have determined to form the joining portion of the sheets by two or more columns of linear joining lines, and provide an interval between the columns of the joining lines. In this way, when the joining portion of the sheets is formed by the two or more columns of linear joining lines, the width of the joining portion widens. For this reason, it is possible to ensure a wide interval between ventilation passages, which are formed when the waist stretchable members contract. Further, when the joining portion is formed by the two or more columns of linear joining lines, and a certain interval is provided between the columns of the joining lines, it is possible to favorably maintain the air permeability and the touch of the joining portion. In addition, the inventors have conceived that the problem of the conventional art may be solved based on the above-described information, and completed the invention. Specifically, the invention has a configuration below.

One aspect of the invention relates to a disposable diaper.

The disposable diaper of the invention is divided into a front body part 1, a rear body part 2, and a crotch portion 3 positioned between the front body part 1 and the rear body part 2 in a longitudinal direction. In addition, in the disposable diaper of the invention, a cushion portion 40 is formed in both or any one of the front body part 1 and the rear body part 2.

The cushion portion 40 includes an internal sheet 41, an external sheet 42, one or a plurality of waist stretchable members 43, and a cushion forming sheet 44.

The external sheet 42 is positioned on a non-skin facing surface of the internal sheet 41.

The one or a plurality of waist stretchable members 43 are fixed by being interposed between the internal sheet 41 and the external sheet 42 while extending in a width direction (X direction) of the disposable diaper.

The cushion forming sheet 44 overlaps both or any one of the internal sheet 41 and the external sheet 42, and is intermittently joined to these sheets 41 and 42 such that joining portions 51 and non-joining portions 52 are alternately continued in the width direction.

Each of the joining portions 51 is formed when two or more columns of linear joining lines 53 extending in the longitudinal direction are arranged.

Herein, when an interval between the joining portions (51) is defined as a first interval (D₁), and an interval between columns of the joining lines (53) within the joining portions (51) is defined as a second interval (D₂), the second interval (D₂) is narrower than the first interval (D₁).

As in the above-described configuration, in the invention, the joining portions 51 are formed when two or more columns of linear joining lines 53 are arranged. Then, a predetermined interval (second interval D₂) is formed between the columns of the linear joining lines 53. In this way, it is possible to improve air permeability and a touch of the joining portions 51. In particular, when an interval between columns of the linear joining lines 53 is set to a certain value or more, a small ventilation passage (space) is expected to be formed between the columns. In addition, when an interval is provided between the columns of the joining lines 53, it is possible to ensure a wide width of the whole joining portion 51. In this way, when the waist stretchable members contract, it is possible to ensure a wide width of the ventilation passage formed in the non-joining portions 52. In addition, when a wide interval between adjacent ventilation passages is held, a contact area of the cushion forming sheet 44 and a skin of a wearer is small, and a wide space is generated between the cushion forming sheet 44 and the skin of the wearer. In this way, it is possible to further enhance air permeability around the belly portion in which the cushion forming sheet 44 is disposed.

In the disposable diaper of the invention, the second interval (D₂) is preferably 15% or more of the first interval (D₁).

As described in the foregoing, when a relatively wide second interval (D₂) is ensured, and when the waist stretchable members 43 contract, a large ventilation passage is expected to be formed between the joining portions 51, and a small ventilation passage is expected to be formed between columns of joining lines 53 within each of the joining portions at the same time. Therefore, the small ventilation passage is positioned between large ventilation passages. In this way, it is possible to solve a convention problem that large ventilation passages press each other, and thus a space hardly widens. In addition, the small ventilation passage is formed within the joining portion 51, and thus it is possible to improve the air permeability and touch of the joining portion 51.

In the disposable diaper of the invention, the second interval (D₂) is preferably 15% or more and 50% or less of the first interval (D₁).

When the second interval (D₂) widens, the large ventilation passage formed between the joining portions 51 becomes smaller. As a result, the air permeability or the touch is hardly maintained within a preferable range as the whole cushion portion 40. For this reason, the second interval (D₂) is preferably 50% or less of the first interval (D₁).

In the disposable diaper of the invention, a width (W) of the joining lines 53 is preferably narrower than the second interval (D₂).

When the width (W) of the joining lines 53 is wider than the second interval (D₂), the air permeability and the touch of the joining portion 51 are impaired. On the other hand, when the width (W) of the joining lines 53 is narrower than the second interval (D₂) as in the above-described configuration, it is possible to maintain excellent air permeability and touch of the joining portions 51.

In the disposable diaper of the invention, the width (W) of the joining lines 53 is preferably 5% or more and 50% or less of the second interval (D₂).

As in the above-described configuration, by setting the width (W) of the joining lines 53 to be 5% or more and 50% or less of the second interval (D₂), it is possible to maintain excellent air permeability and touch of the joining portions 51 while maintaining joining strength for joining sheets by the joining lines 53.

A second aspect of the invention relates to a cushion sheet.

The cushion sheet of the invention is a sheet-shaped member in which one or a plurality of elastic stretchable members is fixed between a plurality of sheet members.

The cushion sheet includes an internal sheet 41, an external sheet 42, one or a plurality of elastic stretchable members 43, and a cushion forming sheet 44.

The elastic stretchable members 43 are fixed by being interposed between the internal sheet 41 and the external sheet 42 while extending in a certain direction.

The cushion forming sheet 44 overlaps both or any one of the internal sheet 41 and the external sheet 42, and is intermittently joined to these sheets 41 and 42 such that joining portions 51 and non-joining portions 52 are alternately continued in a certain direction.

The respective joining portions 51 are formed when two or more columns of linear joining lines 53 extending in a direction intersecting the certain direction (preferably a direction orthogonal to the certain direction) are arranged.

Herein, when an interval between the joining portions (51) is defined as a first interval (D₁), and an interval between columns of the joining lines (53) within the joining portions (51) is defined as a second interval (D₂), the second interval (D₂) is narrower than the first interval (D₁).

### Advantageous Effects of Invention

According to the invention, it is possible to improve air permeability and a tactile property of a joining portion while ensuring a wide interval between ventilation passages formed when waist stretchable members contract in a so-called “flute structure”.

### BRIEF DESCRIPTION OF DRAWINGS

FIG. 1 is an external perspective view of a disposable diaper according to an embodiment of the invention.

FIG. 2 is a developed plan view of the disposable diaper according to the embodiment of the invention.

FIG. 3 is a cross-sectional view taken along Y-Y line illustrated in FIG. 2.

FIGS. 4(a) to 4(c) are schematic views illustrating a structure of a cushion portion. FIG. 4(a) illustrates components included in the cushion portion. FIG. 4(b) illustrates a cross section of the cushion portion in a state in which waist stretchable members are extended. FIG. 4(c) illustrates a cross section of the cushion portion in a state in which the waist stretchable members contract.

FIG. 5 illustrates an example of a joining pattern in the cushion portion.

FIGS. 6(a) to 6(c) illustrate other examples of the joining pattern in the cushion portion.

### DESCRIPTION OF EMBODIMENTS

Hereinafter, embodiments for implementing the invention will be described using drawings. The invention is not restricted to a mode described below, and includes a mode appropriately corrected from the mode below within a clear range by those skilled in the art.

In this specification, a “front body part” refers to a part that covers a belly side (a front of a body) of a wearer when the wearer wears a diaper. In addition, a “crotch portion” refers to a portion that covers a crotch of the wearer when the wearer wears the diaper. In addition, a “rear body part” refers to a part that covers a back side (a rear of the body) of the wearer when the wearer wears the diaper.

In addition, in this specification, a “longitudinal direction” basically refers to a direction (Y axis direction) of connecting a front body part to a rear body part in a disposable diaper. In addition, a “width direction” basically refers to a direction (X axis direction) perpendicular to the longitudinal direction of the disposable diaper.

In addition, in this specification, “A to B” indicates “A or more and B or less”.

### (1. Overall Configuration of Disposable Diaper)

For example, the invention is widely applicable to a known pants-type disposable diaper and a known tape-type disposable diaper. The pants-type disposable diaper is a disposable diaper in which both side portions corresponding to a front body part and a rear body part are joined to each other in advance (for example, Patent Literature 1). In addition, the tape-type disposable diaper is a disposable diaper in which an adhesive tape is attached to both side portions corresponding to a front body part or a rear body part, and the front body part and the rear body part are attached to each other using an adhesive tape at the time of wearing (for example, Patent Literature 3). In addition, examples of the pants-type disposable diaper include an integrated disposable diaper (Patent Literature 1) in which an outer packaging body is one body across the rear body part from the front body part, and a separation-type disposable diaper (Patent Literature 2) in which an outer packaging body of the front body part and an outer packaging body of the rear body part are separated from each other. Hereinafter, content of the invention will be specifically described using, as an example, the separation-type disposable diaper of the pants-type disposable diaper among these various types of disposable diapers.

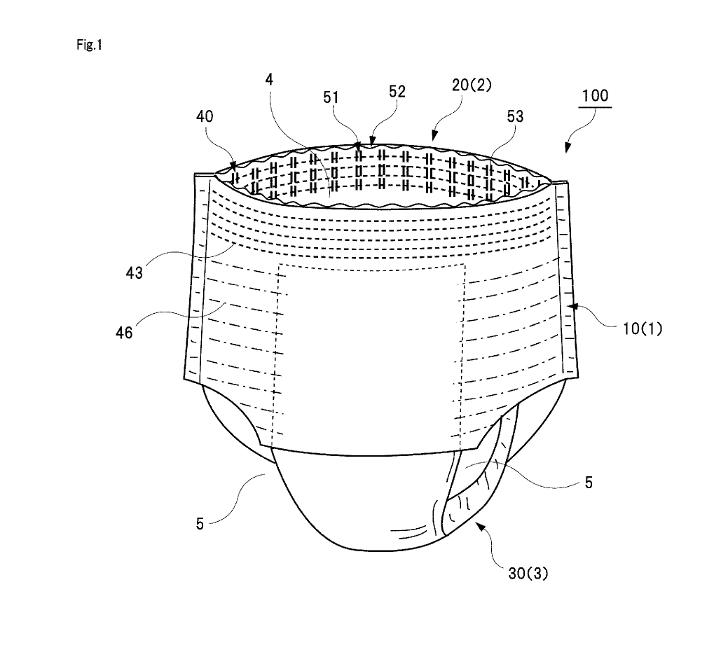

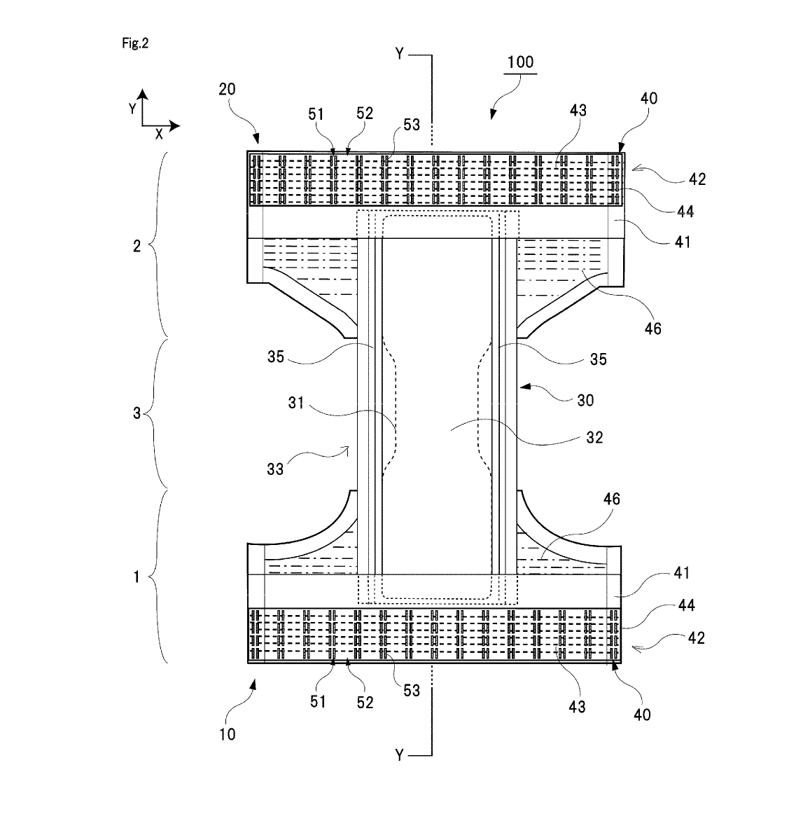

FIG. 1 is a perspective view illustrating an example of a disposable diaper 100 viewed from a front body part side. In addition, FIG. 2 is a developed view illustrating an example of the disposable diaper, and illustrates a state in which the disposable diaper is viewed from a skin facing surface side. As illustrated in the developed view of FIG. 2, the disposable diaper 100 is divided into a front body part 1 coming into contact with a belly portion of a wearer at the time of wearing, a rear body part 2 coming into contact with a back portion of the wearer, and a crotch portion 3 fit to the crotch of the wearer in the longitudinal direction. In the present embodiment, the disposable diaper 100 has the front body part 1 that includes a front body part outer packaging body 10 and the rear body part 2 that includes a rear body part outer packaging body 20. Then, an absorbent main body 30 is bridged in the crotch portion 3 between the front body part outer packaging body 10 and the rear body part outer packaging body 20. That is, one end of the absorbent main body 30 is fixed to the front body part outer packaging body 10, and the other end of the absorbent main body 30 is fixed to the rear body part outer packaging body 20.

The diaper is assembled from a developed state illustrated in FIG. 2 to a state illustrated in FIG. 1 when both end portions of the front body part outer packaging body 10 and the rear body part outer packaging body 20 in the width direction are joined to each other. As illustrated in FIG. 1, a waist opening 4 is formed by an upper edge of the front body part outer packaging body 10 and an upper edge of the rear body part outer packaging body 20. In addition, when the both end portions of the front body part outer packaging body 10 and the rear body part outer packaging body 20 are joined to each other, a leg opening 5 positioned around a leg portion of the wearer at the time of wearing is formed. For this reason, the wearer may wear the disposable diaper 100 by inserting both leg portions from the waist opening 4 and withdrawing each leg portion from the leg opening 5.

In addition, as illustrated in FIG. 2, in the present embodiment, a cushion portion 40 is formed in each of the front body part outer packaging body 10 and the rear body part outer packaging body 20. The cushion portion 40 is formed to extend in the width direction along an end edge that forms the waist opening 4. The cushion portion 40 includes a plurality of waist stretchable members 43. The plurality of waist stretchable members 43 extend in the width direction of the disposable diaper along an end portion of the front body part outer packaging body 10 and an end portion of the rear body part outer packaging body 20 corresponding to an opening edge of the waist opening 4. The plurality of waist stretchable members 43 is fixed in an extended state, and a waist gather is formed around the waist opening 4 when the plurality of waist stretchable members 43 contracts.

In the present embodiment, the cushion portion 40 is provided in each of the front body part outer packaging body 10 and the rear body part outer packaging body 20. However, in the invention, the cushion portion 40 may be provided in at least one of the front body part outer packaging body 10 and the rear body part outer packaging body 20.

### (2. Configuration of Each Portion of Disposable Diaper)

Next, a description will be given of an example of a configuration of each portion of the disposable diaper 100 using drawings.

### (2-1. Cushion Portion)

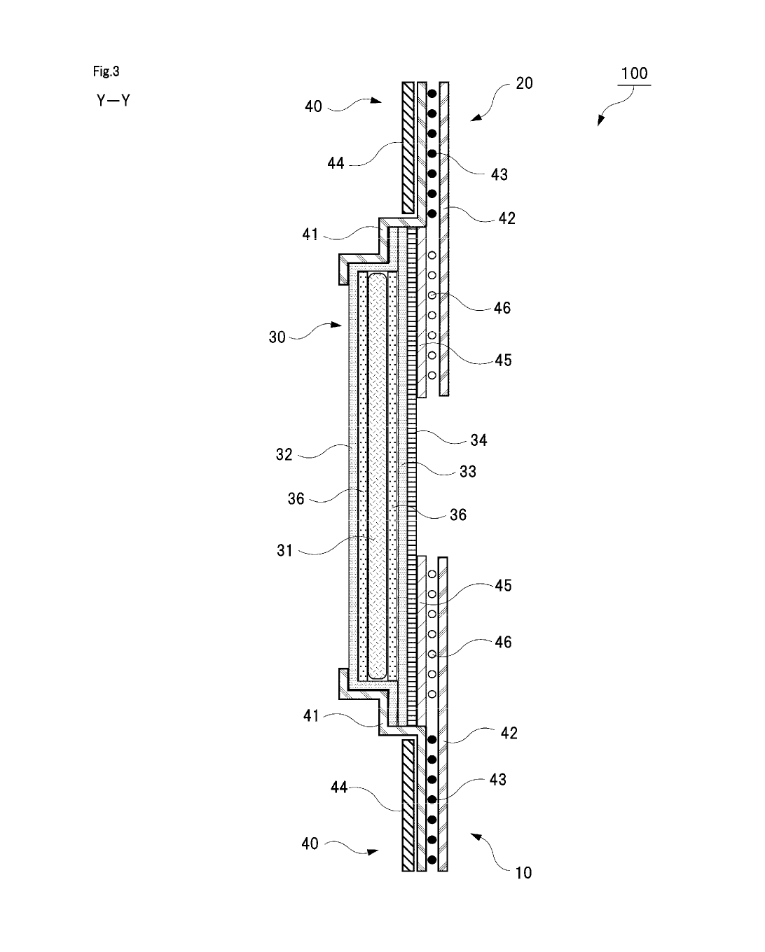

FIG. 3 illustrates a cross-sectional view taken along Y-Y line illustrated in FIG. 2. Even though the disposal diaper is drawn by assigning conceptual thicknesses to various sheet members included in the disposable diaper in the cross-sectional view of FIG. 3, the sheet members included in the disposable diaper are normally significantly thin.

As illustrated in FIG. 3, the front body part outer packaging body 10 and the rear body part outer packaging body 20 (hereinafter collectively referred to as outer packaging bodies 10 and 20) include the cushion portion 40. The cushion portion 40 is a portion that forms a waist gather at the rim of the waist opening 4 of the disposable diaper, and includes a plurality of sheet members and stretchable members.

As illustrated in the cross-sectional view of FIG. 3, the cushion portion 40 includes an internal sheet 41 positioned on a skin facing surface side, an external sheet 42 positioned on a non-skin facing surface side, and a plurality of waist stretchable members 43 fixed between the internal sheet 41 and the external sheet 42. As illustrated in FIG. 3, the internal sheet 41 and the external sheet 42 together form an end edge of the disposable diaper in the longitudinal direction. That is, the internal sheet 41 and the external sheet 42 form an end edge along the waist opening 4 of the disposable diaper. In addition, as illustrated also in a plan view of FIG. 2, the plurality of waist stretchable members 43 extend in parallel in the width direction of the disposable diaper. In addition, the plurality of waist stretchable members 43 is disposed with predetermined intervals in the longitudinal direction of the disposable diaper. In the example illustrated in FIG. 3, the internal sheet 41 and the external sheet 42 are separated from each other. However, the sheets 41 and 42 may be connected and integrally formed. In more detail, one sheet member may be folded back at a waist end edge, thereby setting a portion on the skin facing surface side to the internal sheet 41, and setting a portion on the non-skin facing surface side to the external sheet 42.

The plurality of waist stretchable members 43 is fixed between the internal sheet 41 and the external sheet 42 while being stretched in the width direction. For example, when the plurality of waist stretchable members 43 is fixed, an elongation percentage is preferable in a range of 150% to 400%, 200% to 380%, or 250% to 360%. The “elongation percentage” refers to a value indicating a length of the stretchable member in an extended state when a length (full size) of the stretchable member in a non-extended state (a free state) is set to 100%. Since the waist stretchable member 43 in the extended state is fixed between the internal sheet 41 and the external sheet 42 in this way, a wrinkle (gather) is formed in the internal sheet 41 and the external sheet 42 when the waist stretchable members 43 contract.

When the waist stretchable members 43 is fixed by being interposed between internal sheet 41 and external sheet 42, for example, an adhesive may be applied to the waist stretchable members 43, and the waist stretchable members 43 to which the adhesive is applied may be interposed between the internal sheet 41 and the external sheet 42. In addition, for example, after the adhesive is applied to both or one of the internal sheet 41 and the external sheet 42, the waist stretchable members 43 may be fixed by being interposed between the sheets 41.

Further, in the present embodiment, the cushion portion 40 includes a cushion forming sheet 44 disposed on the skin facing surface side of the internal sheet 41. The cushion forming sheet 44 is a sheet member directly coming into contact with a skin of the wearer of the disposable diaper, and has a function of forming a wrinkle (gather) on the skin facing surface side of the internal sheet 41. The cushion forming sheet 44 may be further disposed on the non-skin facing surface side of the external sheet 42. In addition, the cushion forming sheet 44 overlapping the internal sheet 41 and the cushion forming sheet 44 overlapping the external sheet 42 (not illustrated) may be integrally connected to each other.

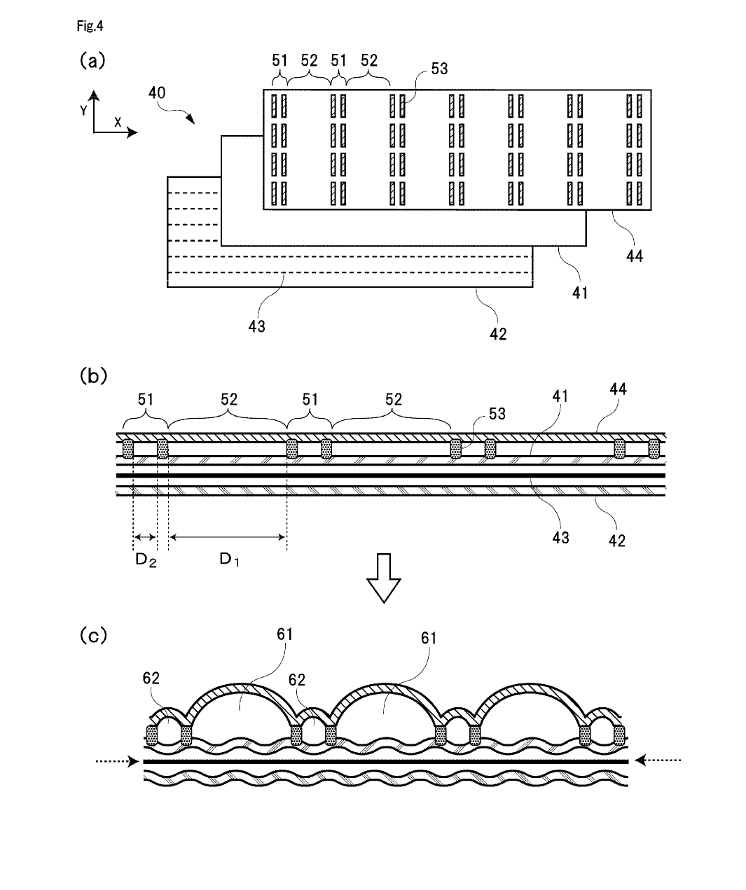

Herein, a description will be given of a structure in which the cushion forming sheet 44 included in the cushion portion 40 forms a wrinkle with reference to FIGS. 4(a) to 4(c). FIGS. 4(a) to 4(c) are schematic views illustrating various components included in the cushion portion 40 by conceptually extracting the components. As illustrated in FIG. 4(a), the cushion portion 40 includes the internal sheet 41, the external sheet 42, the plurality of waist stretchable members 43 fixed between the sheets 41 and 42, and the cushion forming sheet 44 overlapping the internal sheet 41. Although not illustrated, the cushion forming sheet 44 may overlap the external sheet 42, or overlap both the internal sheet 41 and the external sheet 42.

As illustrated in FIG. 4(a), joining portions 51 of the cushion forming sheet 44 and the internal sheet 41 are intermittently formed in the width direction (X axis direction) of the diaper. That is, the cushion forming sheet 44 and the internal sheet 41 are joined to each other such that the joining portions 51 and non-joining portions 52 are alternately continued in the width direction of the diaper. In the joining portions 51, the cushion forming sheet 44 and the internal sheet 41 may be joined to each other using a hot-melt adhesive, etc., or the cushion forming sheet 44 and the internal sheet 41 may be welded by heat sealing or ultrasonic sealing. A pattern of joining lines 53 forming the joining portions 51 will be described below in detail.

FIG. 4(b) illustrates a cross-sectional view in a state in which the external sheet 42, the waist stretchable members 43, the internal sheet 41, and the cushion forming sheet 44 overlap one another in order from a lower side (non-skin contact surface side) of the figure. FIG. 4(b) illustrates a state in which the waist stretchable members 43 do not contract. As illustrated in FIG. 4(b), the waist stretchable members 43 are fixed by being interposed between the internal sheet 41 and the external sheet 42 in the extended state. In addition, the cushion forming sheet 44 is joined to a skin facing surface side of the internal sheet 41 in the joining portions 51 intermittently formed in the width direction.

Next, FIG. 4(c) illustrates an appearance in which the waist stretchable members 43 contract. As illustrated in FIG. 4(c), in response to contraction of the waist stretchable members 43, a wrinkle is formed in the cushion forming sheet 44. That is, when the waist stretchable members 43 contract, the cushion forming sheet 44 is largely separated from the internal sheet 41 in the non-joining portions 52, and thus a large ventilation passage (space) 61 is formed between the cushion forming sheet 44 and the internal sheet 41. Such a structure is referred to as a “flute structure” in this specification. When the large ventilation passage 61 is formed between the intermittently formed joining portions 51 in this way, hardness of the internal sheet 41 interposing the waist stretchable members 43 is hardly directly delivered to the skin of the wearer. In addition, air permeability, flexibility, and a cushioning property may be assigned to the whole waist gather by forming the “flute structure”.

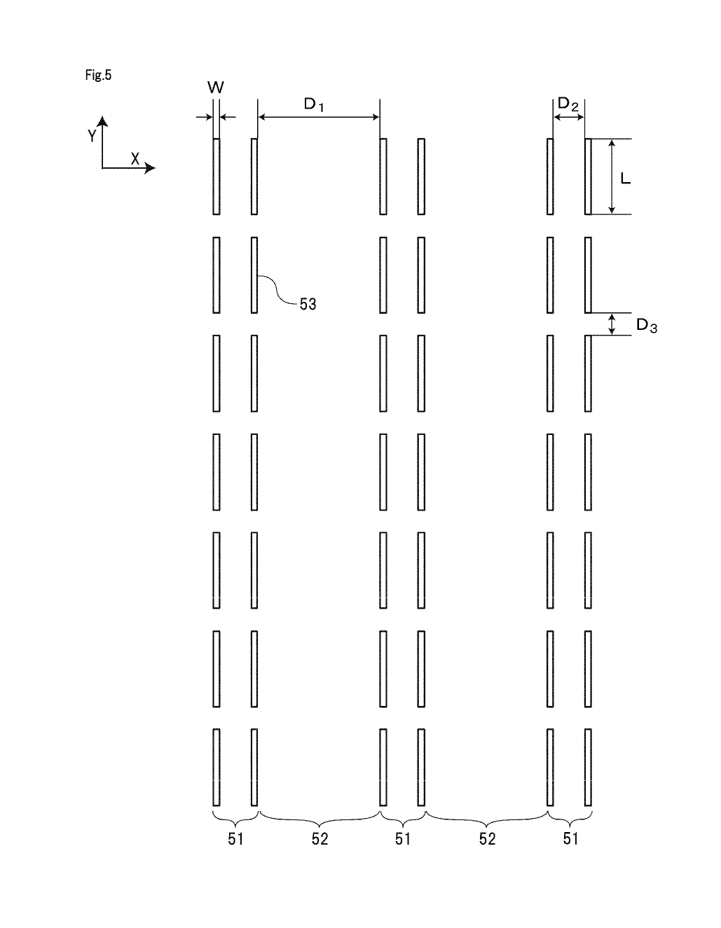

Herein, FIG. 5 illustrates an example of a pattern of the joining portions 51 by enlarging the pattern. As illustrated in FIG. 5, the joining portions 51 and the non-joining portions 52 are alternately formed in the width direction (X direction) of the disposable diaper. In FIG. 5, an interval between the joining portions 51 in the width direction (X direction) is indicated by a symbol D₁. In other words, the interval D₁ may be a width of the non-joining portion 52 in the width direction (X direction). In this specification, the interval indicated by the symbol D₁ is defined as a “first interval”.

In addition, each of the joining portions 51 is formed by a plurality of joining lines 53. Each of the joining lines 53 has a straight line shape extending in the longitudinal direction (Y direction) of the disposable diaper. In FIG. 5, a width of the joining line 53 is indicated by a symbol W, and a length of the joining line 53 is indicated by a symbol L. The length L of the joining line 53 is two times or more, five times or more, or ten times or more the width W of the joining line 53. The joining line 53 is formed in a substantially rectangular shape.

In addition, as illustrated in FIG. 5, two or more columns of linear joining lines 53 are arranged within one joining portion 51. In the example illustrated in FIG. 5, two columns of joining lines 53 are formed within one joining portion 51. A “column” mentioned here refers to a state in which one or a plurality of joining lines 53 is positioned on a (virtual) straight line in the longitudinal direction (Y direction). Within one joining portion 51, the columns of the joining lines 53 are formed with an interval in the width direction (Y direction). In FIG. 5, the interval in the width direction (X direction) between the columns of the joining lines 53 included in one joining portion 51 is indicated by a symbol D₂. In this specification, the interval indicated by the symbol D₂ is defined as a “second interval”.

In addition, in the example illustrated in FIG. 5, a plurality of joining lines 53 belongs to one column. The plurality of joining lines 53 belonging to one column is disposed with an interval in the longitudinal direction (Y direction). In FIG. 5, an interval in the longitudinal direction (Y direction) between columns of joining lines 53 belonging to one column is indicated by a symbol D₃. In this specification, the interval indicated by the symbol D₃ is defined as a “third interval”.

As illustrated in FIG. 5, in this specification, each of the first interval D₁, the second interval D₂, and the third interval D₃ is defined as a distance between edges of adjacent joining lines 53.

Herein, when the “first interval D₁” corresponding to an interval between joining portions 51 is compared with the “second interval D₂” corresponding to an interval between columns of joining lines 53 within one joining portion 51, the second interval D₂ is narrower than the first interval D₁. Specifically, when the first interval D₁ is set to 100%, the second interval D₂ is preferably in a range of 15% to 50%. In addition, the second interval D₂ is preferably in a range of 20% to 45%, and particularly preferably in a range of 22% to 40% or 25% to 35%.

It is considered that three or more columns of joining lines 53 are formed within one joining portion 51, and intervals among the respective columns are different from each other. In this case, the second interval D₂ refers to all intervals among columns of the joining lines 53 within the one joining portion 51. In addition, the second interval D₂ may be rephrased to indicate a widest interval among the intervals among the columns of the joining lines 53 within the one joining portion 51. That is, when three or more columns of joining lines 53 are formed within one joining portion 51, and intervals among the respective columns are different from each other, all the intervals among the respective columns are narrower than the first interval D₁.

As described above, when the second interval D₂ is set to be 15% or more of the first interval D₁, it is considered that a small ventilation passage (space) 62 is formed between columns of joining lines 53 included in the joining portion 51 when the waist stretchable members 43 contract as illustrated in FIG. 4(c). That is, when the waist stretchable members 43 contract, the large ventilation passage 61 is formed between the joining portions 51 (that non-joining portions 52). However, at the same time, the small ventilation passage 62 is formed between columns of joining lines 53 included in the joining portion 51. A height at which the cushion forming sheet 44 rises is smaller in the small ventilation passage 62 than in the large ventilation passage 61. As illustrated in FIG. 4(c), the small ventilation passage 62 having a low height and a narrow width is present between large ventilation passages 61 having a high height and a wide width. In this way, when one or more small ventilation passage 62 is present between the large ventilation passages 61, a wide interval between the large ventilation passages 61 is maintained. That is, when a wide interval between large ventilation passages 61 adjacent to each other is maintained, a contact area of the cushion forming sheet 44 and the skin of the wearer is small, and a wide space is generated between the cushion forming sheet 44 and the skin of the wearer. In this way, it is possible to further enhance air permeability around a belly portion in which the cushion forming sheet 44 is disposed. In addition, when one or more small ventilation passage 62 is present between the large ventilation passages 61, it is possible to prevent the large ventilation passages 61 from coming into contact with and press each other, thereby crushing an internal space thereof. That is, the small ventilation passage 62 exhibits a buffer action of preventing a collision between the large ventilation passages 61. Further, when small ventilation passages 62 are formed at both sides of the large ventilation passage 61, a rising state of the large ventilation passage 61 may be supported by these two small ventilation passages 62. In this way, the large ventilation passage 61 is supported by the small ventilation passages 62, and thus the rising state thereof is hardly destroyed, and a cushioning property is improved. That is, the small ventilation passages 62 exhibit a support action of supporting the large ventilation passage 61. When the small ventilation passage 62 is provided between the large ventilation passages 61 (in other words, the large ventilation passage 61 is provided between the small ventilation passages 62), it is possible to more effectively exhibit functions of the large ventilation passage 61 (improvements in air permeability, flexibility, and a cushioning property). In particular, when the joining lines 53 have linear shapes, for example, the small ventilation passage 62 is easily formed between columns of the joining lines 53 when compared to a case in which circular joining points, etc. are formed instead of the joining lines 53. For this reason, the joining lines 53 may preferably have linear shapes.

In addition, when columns of the joining lines 53 are formed at an interval within the joining portion 51, air permeability or flexibility of the joining portion 51 is improved. That is, it may be considered that the joining portion 51 is formed by one column of thick joining lines or dense joining points in order to ensure a wide interval between the large ventilation passages 61. However, since the joining portion 51 corresponds to a region obtained by joining sheet members using an adhesive, thermal fusion bonding, etc., air permeability or flexibility of the joining portion 51 is degraded when the joining portion 51 is formed by the thick joining lines or the dense joining points. In contrast, air permeability and flexibility of the joining portion 51 may be prevented from being degraded when one joining portion 51 is formed by a plurality of columns of joining lines 53 at an interval as in the pattern illustrated in FIG. 5.

Besides, when the second interval D₂ exceeds 50% of the first interval D₁, the small ventilation passage 62 becomes excessively large. That is, when the second interval D₂ exceeds 50% of the first interval D₁, there is a concern that the large ventilation passage 61 and the small ventilation passage 62 adjacent to each other may interfere in each other to crush spaces thereof. In this case, there is a concern that the functions of the large ventilation passage 61 (improvements in air permeability, flexibility, and the cushioning property) may be inhibited by the small ventilation passage 62. In addition, as described in the foregoing, the small ventilation passage 62 has the function of ensuring air permeability by maintaining the wide interval between the large ventilation passages 61, the buffer action of preventing the collision between the large ventilation passages 61, and the support action of supporting the large ventilation passage 61. However, when the second interval D₂ exceeds 50% of the first interval D₁, the above-described three actions by the small ventilation passage 62 are not effectively exhibited. In this regard, in the invention, the second interval D₂ is preferably 50% or less or 40% or less of the first interval D₁, and particularly preferably 30% or less of the first interval D₁.

In addition, a height at which the small ventilation passage 62 rises is preferably 50% or less, 40% or less, or 30% or less of a height at which the large ventilation passage 61 rises. Specifically, when a height of the large ventilation passage 61 is set to 100%, a height of the small ventilation passage 62 is preferably in a range of 10% to 50%, and particularly preferably in a range of 12% to 45%, 14% to 40%, or 15% to 30%. The heights of the small ventilation passage 62 and the large ventilation passage 61 refer to heights obtained when the waist stretchable members 43 in a non-extended state (a free state). The small ventilation passage 62 is formed between two large ventilation passages 61. When the height of the small ventilation passage 62 exceeds 50% of the height of the large ventilation passage 61, air permeability between the large ventilation passages 61 is impaired. In addition, when the height of the small ventilation passage 62 exceeds 50% of the height of the large ventilation passage 61, the above-described three actions by the small ventilation passage 62 are not effectively exhibited. Then, a purpose of forming the small ventilation passage 62 between the large ventilation passages 61 almost disappears. For this reason, the height at which the small ventilation passage 62 rises is preferably half or less the height at which the large ventilation passage 61 rises.

In addition, as illustrated in FIG. 5, the width W of the joining line 53 is preferably the second interval D₂ or less. In particular, the width W of the joining line 53 is preferably in a range of 5% to 50%, and particularly preferably in a range of 7% to 30% or 10% to 20% with respect to the second interval D₂. When the width W of the joining line 53 exceeds 50% of the second interval D₂, there is a concern that a space of the large ventilation passage 61 may decrease in size, or the small ventilation passage 62 may not be formed. Meanwhile, when the width W of the joining line 53 is less than 5% of the second interval D₂, adhesion between the sheets decreases, and thus there is a concern that the sheets may easily peel off. For this reason, the width W of the joining line 53 is preferably in the range of 5% to 50% with respect to the second interval D₂ in order to maintain air permeability and the cushioning property of the cushion portion 40.

In addition, for example, the third interval D₃ illustrated in FIG. 5 may be set to a range of 10% to 50%, 20% to 40%, or 25% to 35% with respect to the length L of the joining line 53 in order to maintain joining strength and air permeability of the joining portion 51.

With regard to a joining pattern illustrated in FIG. 5, a description will be given of actual numerical values of various dimensions. For example, D₁ is in a range of 7.0 to 9.0 mm, D₂ is in a range of 1.5 to 3.5 mm, W is in a range of 0.2 to 1.0 mm, L is in a range of 3 to 7 mm, and D₃ is in a range of 0.5 to 3 mm. However, specific numerical values of various dimensions are not restricted thereto, and appropriate design changes are allowed.

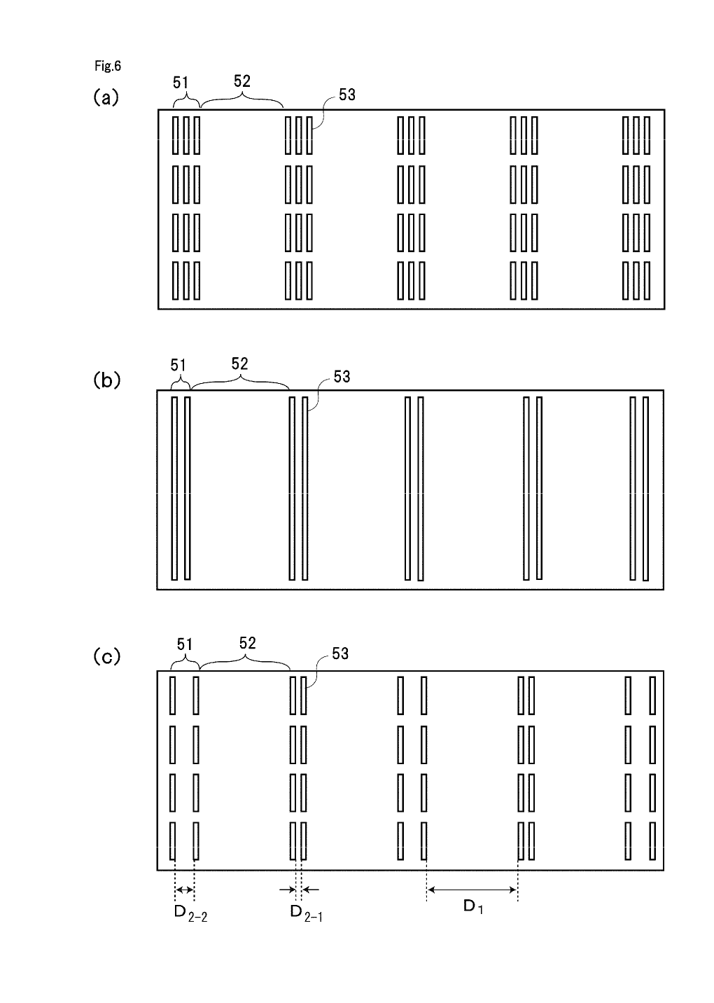

FIGS. 6(a) to 6(c) illustrate other examples of the joining pattern. As illustrated in FIG. 6(a), three columns of joining lines 53 may be included in one joining portion 51. In addition, although not illustrated, four or more columns of joining lines 53 may be included in one joining portion 51. In addition, as illustrated in FIG. 6(b), a column of the joining line 53 may be formed by only one joining line 53.

In addition, as illustrated in FIG. 6(b), intervals between columns of joining lines 53, that is, all the second intervals may be equal to each other. In the examples illustrated in FIGS. 6(a) to 6(c), two types of second intervals are present. For example, when the two types of second intervals are defined as a 2-1th interval D₂₋₁ and a 2-2th interval ₂₋₂, the 2-1th interval D₂₋₁ is narrower than the 2-2th interval ₂₋₂. In this case, as described above, each of the 2-1th interval D₂₋₁ and the 2-2th interval ₂₋₂ preferably falls within a range of 15% to 50% with respect to the first interval D₁. However, the scope of the invention may not be departed from when any one (narrower one) of the 2-1th interval D₂₋₁ and the 2-2th interval ₂₋₂ falls within the range of 15% to 50% with respect to the first interval D₁. In addition, although not illustrated, it is possible to consider a case in which two types or more of first intervals D₁ are present. In this case, the second interval may be set within the range of 15% to 50% using a widest interval among a plurality of types of first intervals D₁ as a reference interval.

Besides, each of the internal sheet 41, the external sheet 42, and the cushion forming sheet 44 is preferably formed using a material such as a nonwoven fabric having an excellent touch and air permeability. For example, a nonwoven fabric manufactured using a spunbond method, an air-through method, or an air-laid method may be used.

In addition, it is preferable to apply filiform elastic gum or belt-like elastic gum as the waist stretchable members 43. A material such as styrene rubber, olefin rubber, urethane rubber, ester rubber, polyurethane, polyethylene, polystyrene, styrene-butadiene, silicone, polyester, etc. may be used as such a rubber material.

In addition, in the present embodiment, the internal sheet 41 extends toward the crotch portion 3 of the diaper. Further, a portion of the internal sheet 41 overlaps an upper part (skin facing surface) of an end portion of the absorbent main body 30 in the longitudinal direction. When the portion of the internal sheet 41 is overlapped with the absorbent main body 30, the absorbent main body 30 is firmly fixed to the outer packaging bodies 10 and 20 and is unlikely to be removed.

In addition, the outer packaging bodies 10 and 20 may further include an inner sheet 45 and a stretchable tammy member 46. In more detail, as illustrated in FIG. 3, in the present embodiment, the external sheet 42 extends toward the crotch portion 3, and a portion thereof is positioned on the non-skin facing surface of the absorbent main body 30. Herein, the inner sheet 45 is disposed on the skin facing surface side of the extending portion of the external sheet 42. Further, one or a plurality of stretchable tammy members 46 is fixed by being interposed between the extending portion of the external sheet 42 and the inner sheet 45. As with the waist stretchable members 43, the stretchable tammy member 46 is fixed between the extending portion of the external sheet 42 and the inner sheet 45 in an extended state in the width direction of the diaper.

### (2-3. Absorbent Main Body)

As illustrated in FIG. 2 and FIG. 3, the absorbent main body 30 is held while being bridged between the front body part outer packaging body 10 and the rear body part outer packaging body 20, is positioned in the crotch portion of the wearer at the time of wearing the disposable diaper 100, and absorbs and holds a liquid such as urine excreted by the wearer. The absorbent main body 30 is disposed across the front body part 1 and the rear body part 2 around the crotch portion 3 of the disposable diaper. As illustrated in FIG. 2 and FIG. 3, the absorbent main body 30 includes an absorber 31, a top sheet 32, a back sheet 33, a cover sheet 34, and a pair of solid gathers 35.

The absorber 31 is a member for absorbing a liquid such as urine, and holding the absorbed liquid. The absorber 31 is disposed between the liquid-permeable top sheet 32 and the liquid-impermeable back sheet 33. The absorber 31 has a function of absorbing a liquid penetrating the top sheet 32, and is made of an absorbent material. A known material may be employed as the absorbent material contained in the absorber 31. For example, a flap pulp, a super absorbent polymer, or a hydrophilic sheet may be used as the absorbent material. The absorber 31 may be enclosed by a core wrap sheet 36. Tissue paper, absorbent paper, a nonwoven fabric subjected to a hydrophilic treatment, etc. may be appropriately used as the core wrap sheet 36.

The top sheet 32 is a member that directly comes into contact with a skin of the crotch portion of the wearer, and allows a liquid such as urine to penetrate to the absorber 31. For this reason, the top sheet 32 is made of a liquid-permeable material having high flexibility. In addition, the top sheet 32 is disposed to cover a skin contact surface side of the absorber 31. Examples of the liquid-permeable material contained in the top sheet 32 include a fabric, a nonwoven fabric, or a porous film. In addition, for example, a material obtained by performing a hydrophilic treatment on a fiber of a thermoplastic resin such as polypropylene, polyethylene, polyester, or nylon, and then forming a nonwoven fabric using the fiber may be used as the top sheet 32.

The back sheet 33 is a member for preventing the liquid, which penetrates the top sheet 32 and is absorbed by the absorber 31, from leaking to the outside of the diaper. For this reason, the back sheet 33 is made of a liquid-impermeable material. The back sheet 33 covers the absorber 31 from the non-skin contact surface side to prevent a liquid leakage from a bottom surface of the absorber 31. An example of the impermeable material contained in the back sheet 33 corresponds to a liquid-impermeable film made of polyethylene resin. In particular, it is preferable to use a microporous polyethylene film in which a plurality of minute holes having a size in a range of 0.1 to 4 μm is formed.

The cover sheet 34 is a member for reinforcing the back sheet 33 and improving a touch thereof. The cover sheet 34 is bonded to the non-skin contact surface side of the back sheet 33. A woven fabric or a nonwoven fabric is used as a material contained in the cover sheet 34. In particular, it is preferable to use a nonwoven fabric or a wet-laid nonwoven fabric made of a thermoplastic resin such as polyethylene, polypropylene, or polyester as the material contained in the cover sheet 34.

The pair of solid gathers 35 is a member that rises along both side edges of the absorber 31, and prevents a side leakage of urine excreted by the wearer. A solid gather stretchable material is disposed at a distal end of each of the solid gathers 35, and rises in a skin contact direction of the wearer when the stretchable material contracts. For this reason, the pair of solid gathers 35 serves as a urine leakproof wall, and prevents a side leakage in which urine penetrating the top sheet 32 or urine failing to be absorbed by the absorber 31 leaks from an opening around a leg portion of the disposable diaper. The solid gathers 35 may employ a known configuration used for a conventional disposable diaper. For example, the solid gathers 35 may be formed by interposing and fixing the solid gather stretchable material in an extended state between layers of a water-repellent sheet. For example, a nonwoven fabric sheet obtained using a manufacturing method such as card embossing, spunbond, etc. may be used as the water-repellent sheet. In particular, it is preferable to use a nonwoven fabric such as SMS, SMMS, etc. having a high waterproofing property.

Hereinbefore, in this specification, a description has been given of the embodiments of the invention with reference to the drawings to express content of the invention. However, the invention is not restricted to the above embodiments, and includes a modified mode or an improved mode clear to those skilled in the art based on information described in this specification.

For example, in the invention, a cushioning property may be assigned by manufacturing the cushion sheet illustrated in FIGS. 4(a) to 4(d) as a separate body from the disposable diaper, and bonding the cushion sheet around the waist of the disposable diaper.

In more detail, as illustrated in FIG. 4(a), the cushion sheet is a sheet-shaped member in which one or a plurality of elastic stretchable members is fixed between a plurality of sheet members. When the plurality of elastic stretchable members is present, the respective elastic stretchable members are disposed in parallel. The cushion sheet includes the internal sheet 41, the external sheet 42, the one or the plurality of elastic stretchable members 43, and the cushion forming sheet 44. The elastic stretchable members 43 are fixed by being interposed between the internal sheet 41 and the external sheet 42 in an extended state in a certain direction. The cushion forming sheet 44 overlaps both or any one of the internal sheet 41 and the external sheet 42, and is intermittently joined to these sheets 41 and 42 such that the joining portions 51 and the non-joining portions 52 are alternately continued in the certain direction. The respective joining portions 51 are formed when two or more columns of linear joining lines 53 extending in a direction intersecting the certain direction (preferably a direction orthogonal to the certain direction) are arranged. Herein, when an interval between the joining portions 51 is defined as the first interval (D₁), and an interval between columns of joining lines 53 within the joining portion 51 is defined as the second interval (D₂), the first interval (D₂) is narrower than the first interval (D₁). The second interval (D₂) is preferably 15% or more and 50% or less of the first interval (D₁).

As described in the foregoing, a cushioning property may be easily assigned to an existing disposable diaper by manufacturing a cushion sheet separately from the disposable diaper. For this reason, manufacturing of only the cushion sheet is a preferable mode of the invention.

### INDUSTRIAL APPLICABILITY

The invention is preferably applicable to a disposable diaper manufacturing industry.

### REFERENCE SIGNS LIST

1 . . . front body part
2 . . . rear body part
3 . . . crotch portion
4 . . . waist opening
5 . . . leg opening
10 . . . front body part outer packaging body
20 . . . rear body part outer packaging body
30 . . . absorbent main body
31 . . . absorber
32 . . . top sheet
33 . . . back sheet
34 . . . cover sheet
35 . . . solid gather
36 . . . core wrap sheet
40 . . . cushion portion
41 . . . internal sheet
42 . . . external sheet
43 . . . waist stretchable member
44 . . . cushion forming sheet
45 . . . inner sheet
46 . . . stretchable tammy member
51 . . . joining portion
52 . . . non-joining portion
53 . . . linear joining line
61 . . . large ventilation passage
62 . . . small ventilation passage

1. A disposable diaper divided into a front body part (1), a rear body part (2), and a crotch portion (3) positioned between the front body part (1) and the rear body part (2) in a longitudinal direction, a cushion portion (40) being formed in both or any one of the front body part (1) and the rear body part (2),
wherein the cushion portion (40) includes:
an internal sheet (41);
an external sheet (42) positioned on a side of a non-skin facing surface of the internal sheet (41);
one or a plurality of waist stretchable members (43) fixed by being interposed between the internal sheet (41) and the external sheet (42) while extending in a width direction of the disposable diaper; and
a cushion forming sheet (44) overlapping both or any one of the internal sheet (41) and the external sheet (42), the cushion forming sheet (44) being intermittently joined thereto such that joining portions (51) and non-joining portions (52) are alternately continued in the width direction,
each of the joining portions (51) is formed when two or more columns of linear joining lines (53) extending in the longitudinal direction are arranged, and
when an interval between the joining portions (51) is defined as a first interval (D₁), and an interval between columns of the joining lines (53) within the joining portions (51) is defined as a second interval (D₂), the second interval (D₂) is narrower than the first interval (D₁).

2. The disposable diaper according to claim 1, wherein the second interval (D₂) is 15% or more of the first interval (D₁).

3. The disposable diaper according to claim 1, wherein the second interval (D₂) is 50% or less of the first interval (D₁).

4. The disposable diaper according to claim 1, wherein a width (W) of the joining lines (53) is narrower than the second interval (D₂).

5. The disposable diaper according to claim 4, wherein the width (W) of the joining lines (53) is 5% or more and 50% or less of the second interval (D₂).

6. A cushion sheet in which one or a plurality of elastic stretchable members is fixed between a plurality of sheet members, the cushion sheet comprising:
an internal sheet (41);
an external sheet (42);
one or a plurality of elastic stretchable members (43) fixed by being interposed between the internal sheet (41) and the external sheet (42) while extending in a certain direction; and
a cushion forming sheet (44) overlapping both or any one of the internal sheet (41) and the external sheet (42), the cushion forming sheet (44) being intermittently joined thereto such that joining portions (51) and non-joining portions (52) are alternately continued in the certain direction,
wherein each of the joining portions (51) is formed when two or more columns of linear joining lines (53) extending in a direction intersecting the certain direction are arranged, and
when an interval between the joining portions (51) is defined as a first interval (D₁), and an interval between columns of the joining lines (53) within the joining portions (51) is defined as a second interval (D₂), the second interval (D₂) is narrower than the first interval (D₁).
